(19) United States
(12) Patent Application Publication
(10) Pub. No.: US 2004/0115279 A1
(43) Pub. Date: Jun. 17, 2004

(54) Microfabrication of polymer microparticles

(75) Inventors: Derek J. Hansford, Columbus, OH (US); Jingjiao Guan, Columbus, OH (US)

Correspondence Address: CALFEE, HALTER & GRISWOLD, LLP, 1110 FIFTH THIRD CENTER, 21 EAST STATE STREET, COLUMBUS, OH, 43215-4243, US

(73) Assignee: The Ohio State University

(21) Appl. No.: 10/656,661

(22) Filed: Sep. 5, 2003

Related U.S. Application Data
(60) Provisional application No. 60/408,557, filed on Sep. 6, 2002.

(51) Int. Cl. A61K 9/14; B27N 3/00

(52) U.S. Cl. 424/489; 264/109

(57) ABSTRACT

A system and method for creating polymer microparticles for use in drug delivery and other applications. The components of the exemplary system include a micro-stamp having micro-contours or micro-structures, a substrate, and a sacrificial layer of material coating the slide. The method includes the steps of coating the face of stamp with a thin layer of polymer to cover the micro-structures of the stamp, contacting the coated face of the stamp with the coated substrate to transfer polymer from the micro-structures of the stamp to the slide to create free-standing polymer microparticles, and dissolving the sacrificial layer covering the substrate to release the microparticles into solution. The microparticles fabricated by this method typically exhibit well-defined geometries that correspond to the micro-structures of the stamp.

### CROSS-REFERENCE TO RELATED APPLICATIONS

This patent application claims the benefit of U.S. Provisional Patent Application Serial No. 60/408,557 filed on Sep. 6, 2002 and entitled “Microfabrication of Polymer Microparticles,” the disclosure of which is incorporated as if fully rewritten herein.

### STATEMENT REGARDING FEDERALLY FUNDED R&D

This invention was made with government support under Agreement Number F30602-00-2-0613 awarded by the Defense Advanced Research Projects Agency (DARPA), and the Air Force Research Laboratory (AFRL), Air Force Materiel Command, USAF. The government has certain rights in the invention.

### TECHNICAL FIELD OF THE INVENTION

The present invention relates generally to methods and techniques for fabricating microparticles for a use in scientific and/or medical applications and more specifically to a microfabrication method for creating polymer microparticles having certain geometric, structural, and compositional characteristics.

### BACKGROUND OF THE INVENTION

Polymer microparticles are useful for a variety of applications, including biological and medical analysis, drug delivery, bio-separation, and clinical diagnosis. Polymer microparticles may take numerous forms such as, for example, microbeads, microspheres, microbubbles, and/or microcapsules. A variety of manufacturing and/or fabrication methodologies have been developed and applied to create such microparticles. These known methods include spray drying, phase separation, and emulsification. Despite the demonstrated effectiveness of these techniques, the microparticles produced by these methods are typically limited to shapes that are spherical or substantially spherical. Furthermore, the size of the particles produced by these methods is often widely distributed.

While spherical microparticles are useful for certain applications such as drug delivery, non-spherical particles may prove to have more desirable characteristics. Substantially flat microparticles possess a comparatively large surface area and, as will be appreciated by those skilled in the art, may be more suitable for cell or tissue binding applications. Furthermore, discrete control of particle geometry, and other characteristics such as particle thickness, may facilitate more precise bio-analysis and controlled drug delivery because the shape of a particle can be tailored to function more effectively under certain predefined conditions. Thus, there is a need to identify an effective method for fabricating substantially flat microparticles having desired geometries, structural characteristics, and/or other characteristics that provide enhanced functionality in various applications.

Microfabrication techniques conventionally used for making integrated circuits have recently been utilized to create microparticles by combining silicon dioxide or polymethylmethacrylate (PMMA) and a photo-sensitive polymer. These techniques can be used to create microparticles having a precise shape, uniform size and specifically designed structures and surface chemistries, thereby making them suitable for use as drug-carrying vehicles. However, these techniques are limited in that they (i) require the use of photolithography to create every particle and (ii) are compatible with only certain materials. Moreover, the rigorous conditions, including highly aggressive solutions and elevated temperatures, which are used to release fabricated microparticles into solution may damage fragile compounds that have been incorporated into the microparticles. Thus, there are significant limitations to using known photolithographic techniques for microfabrication of shaped microparticles.

An alternative to conventional photolithographic techniques is soft-lithography. Soft lithography is a collective term that refers to a group of non-photolithographic microfabrication techniques that employ elastomeric stamps having certain three dimensional relief features to generate micro-structures and even nano-structures. A more detailed description of soft lithography is found in Xia and Whitesides, Annual Review of Materials Science 28: 153-84 (1998) incorporated herein by reference. Thus, there is a need to utilize such alternate microfabrication techniques to create polymer microparticles having certain desired geometries.

### SUMMARY OF THE INVENTION

These and other deficiencies of the prior art are overcome by the present invention, which provides a system and methods for using common thermoplastic polymers to prepare thin-film microparticles having well-defined lateral geometries and other desired characteristics. The components of the exemplary system include a PDMS stamp having micro-contours or micro-structures, a substrate, and a sacrificial layer of material coating the substrate. The basic method includes the steps of coating the face of stamp with a thin layer of polymer to cover the micro-structures of the stamp, contacting the coated face of the stamp with the coated glass slide to transfer polymer from the micro-structures of the stamp to the slide to create free-standing polymer microparticles, and dissolving the sacrificial layer covering the substrate to release the microparticles into solution. The microparticles fabricated by this method typically exhibit well-defined geometries that correspond to the micro-structures of the stamp.

Further advantages of the present invention will become apparent to those of ordinary skill in the art upon reading and understanding the following detailed description of the preferred embodiments.

### BRIEF DESCRIPTION OF THE DRAWINGS

The accompanying drawings, which are incorporated into and form a part of the specification, schematically illustrate one or more exemplary embodiments of the invention and, together with the general description given above and detailed description of the preferred embodiments given below, serve to explain the principles of the invention.

FIGS. 1a-d illustrate graphically the system components and stepwise method of the embodiment of the microfabrication technique of the present invention that utilizes the micro-pillar surface structures of a PDMS stamp.

FIGS. 2a-f illustrate graphically the system components and stepwise method of the embodiment of the microfabrication technique of the present invention that utilizes the micro-well surface structures of a PDMS stamp.

FIGS. 3a-e illustrate graphically the system components and stepwise method of the embodiment of the microfabrication technique of the present invention that utilizes a discontinuous wetting technique to fill the well surface structure of a PDMS stamp.

FIGS. 4a-h illustrate graphically the system components and stepwise method of the embodiment of the microfabrication technique of the present invention that utilizes the multiple filling process to produce multi-layer particles within the well surface structures of a PDMS stamp.

FIG. 5a is an optical micrograph of polymer microparticles attached to a substrate, showing replication of the geometry of the micro-pillars found on the face of the PDMS stamp.

FIG. 5b is an optical micrograph of the microparticles of FIG. 5a released from the substrate and floating freely in solution.

FIG. 6a is an optical micrograph of polymer microparticles attached to a substrate, showing replication of the geometry of the micro-wells found on the face of the PDMS stamp.

FIG. 6b is an optical micrograph of the microparticles of FIG. 6a released from the substrate and floating freely in solution.

FIG. 7 is an optical micrograph of microparticles fabricated using the discontinuous wetting technique floating freely in solution after release from the substrate.

FIG. 8 is an optical micrograph of 3-layer microparticles fabricated using the multiple layer technique floating in solution, showing the middle layer of FSPAN swollen, but confined between the two layers of PPMA.

### DETAILED DESCRIPTION OF THE INVENTION

The present invention provides a basic system and several alternate methods for using common thermoplastic polymers to prepare thin-film microparticles that exhibit well-defined lateral geometries and other desired characteristics. The exemplary embodiment of this system includes a polydimethyl siloxane stamp having micro-contours or micro-structures, a substrate, and a sacrificial layer of material coating the substrate. As described below, stamps with both isolated protruding structures and recessed structures can be used to create polymer microparticles using the system and methods of this invention.

The exemplary methods of the present invention primarily utilize the polymer polypropyl methacrylate (“PPMA”), although other common polymers such as, for example, polylactic-co-glycolic acid, polycaprolactone, polymethyl methacrylate, and polystyrene have been successfully demonstrated with this system. Furthermore, the general methods disclosed herein are easily extendable to most polymers, and thermoplastic polymers, in particular. The exemplary system also utilizes polydimethyl siloxane (PDMS) stamps having two different types of surface structures: (i) micro-pillars, which comprise square-like members with rounded corners protruding from the face of the stamp, and (ii) micro-wells which comprise square-like recessed areas formed between the micro-pillars on the face of the stamp. As will be appreciated by those skilled in the art of soft-lithography, PDMS stamps are typically created from molds. The dimensions of PDMS stamps are typically about 1.0 cm×1.0 cm, although much larger stamps can be created for large-scale manufacturing.

In exemplary embodiment, the sacrificial layer component typically consists of polyvinyl alcohol (PVA) due to its solubility in water and its high melting temperature. However, other materials that exhibit solubility in water and relatively low solubility in other solvents may be suitable for the disclosed system. In other embodiments, water-soluble inks, glucose, chitosan, and polyethylene glycol (PEG) are utilized. In the exemplary methods, the substrate that the sacrificial layer is deposited on is typically a glass slide; however, other substantially flat, smooth, non-porous materials may be used.

I. Micro-Pillar Printing Method

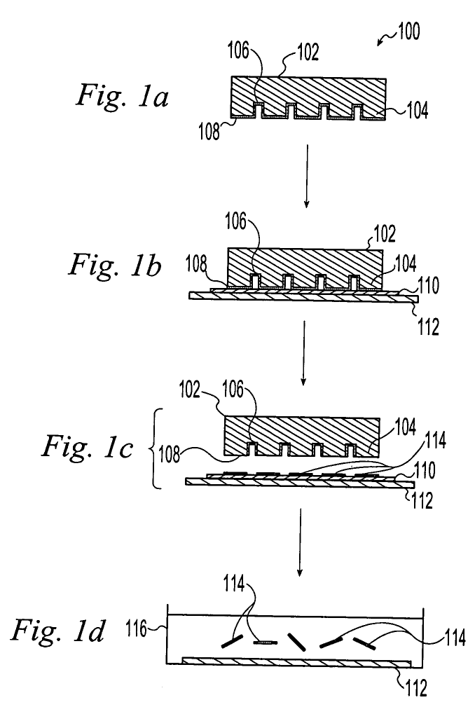

With reference now to FIGS. 1a-d, a first embodiment of microfabrication system 100 includes a stamp 102, a substrate 112, and a water-soluble sacrificial layer 110. Utilizing system 100, microparticles 114 are fabricated according to the following exemplary method:

(i) dip stamp 102 into a 5.8 wt % PPMA/acetone solution to form a thin, continuous layer 108 of PPMA on the face of the stamp (see FIG. 1a) which covers its contours, i.e., micro-pillars 104 and micro-wells 106;

(ii) using a cotton swab or other suitable applicator, brush a 1.5 wt % aqueous solution of polyvinyl alcohol (PVA) onto the surface of a glass slide (substrate 112) to form a thin film which will serve as sacrificial layer 110 (see FIG. 1b);

(iii) place stamp 102 on substrate 112 with the polymer-coated face touching the surface of the slide and sacrificial layer 110 and place a solid weight on top of the stamp, creating a pressure of about 320 Pa, to ensure a complete conformal contact between stamp 102 and substrate 112;

(iv) place the weight, stamp, and slide on a hot plate at about 110° C. for about ten seconds;

(v) peel stamp 102 away from substrate 112 leaving the polymer microparticles attached to sacrificial layer 110 (see FIG. 1c);

(vi) place substrate 112 into water-filled reservoir 116 to dissolve sacrificial layer 110 and release microparticles 114 into solution (see FIG. 1d); and (optionally)

(vii) retrieving microparticles from solution by means of desiccation, filtration, or any other suitable method.

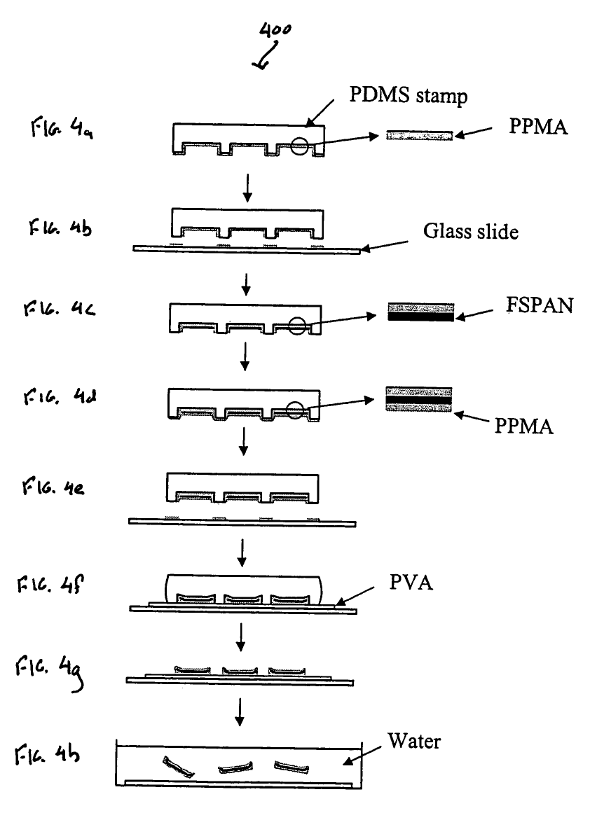

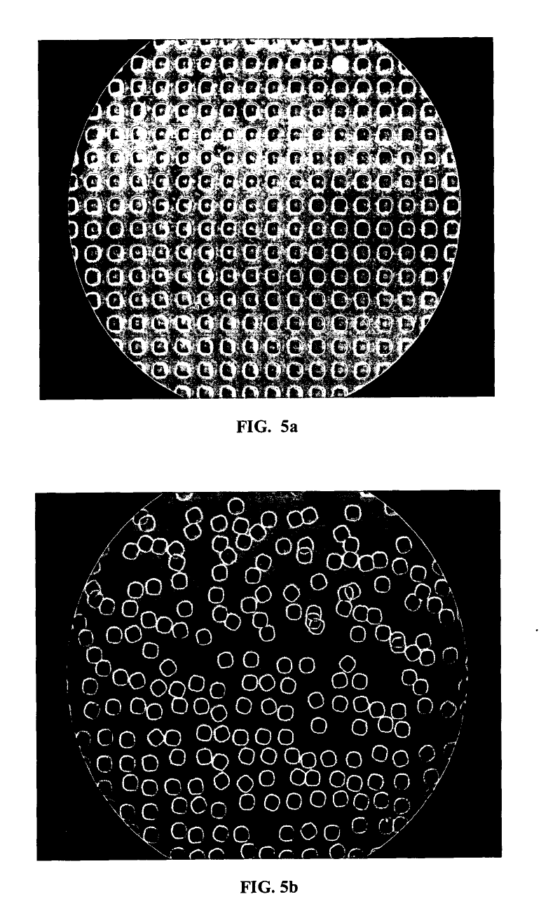

With reference to FIG. 5a, the width and height of micro-pillars 104 is about 30 μm by about 3.7 μm, respectively, and the resultant particles have a width of about 30 μm and thickness of about 650 nm. FIG. 5a is an optical micrograph of microparticles 114 on substrate 112 showing replication of the structures of the micro-pillars, namely the generally square shape with rounded corners. FIG. 5b is an optical micrograph of microparticles 114 released into a solution of water after sacrificial layer 112 has been dissolved.

II. Micro-Well Printing Method

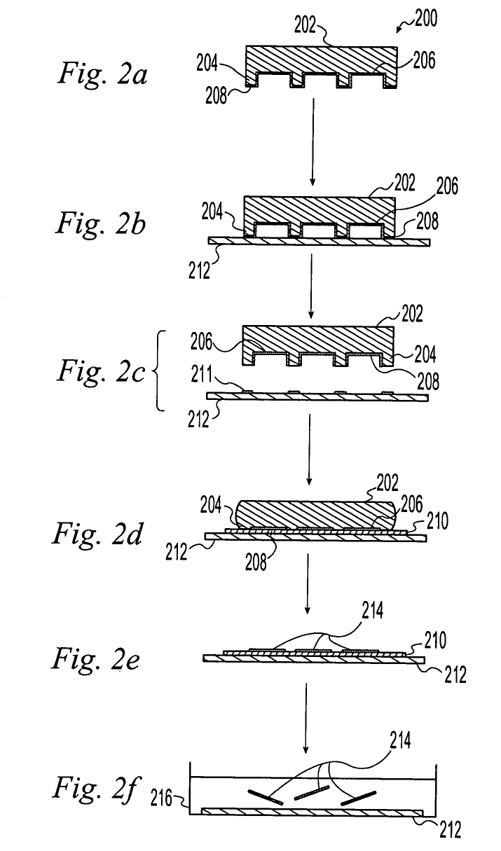

With reference to FIGS. 2a-f a second embodiment of this invention, microfabrication system 200, uses a stamp 202, a substrate 212, and a water-soluble sacrificial layer 210 to create polymer microparticles 214. Utilizing system 200, microparticles 214 are fabricated according to the following exemplary method:

(i) stamp 202 is dipped into a 2.5 wt % PPMA/acetone solution to form a thin, continuous layer 208 of PPMA on the face of the stamp (see FIG. 2a) and covering its contours, i.e., micro-pillars 204 and micro-wells 206;

(ii) place the polymer-coated stamp on a glass slide 212 (see FIG. 2b) and apply pressure of about 550 Pa to the stamp using a solid weight to induce a full conformal contact between the polymer on the raised regions of the stamp and the glass slide across the entire face of the stamp (note: care should be taken to not apply excessive pressure resulting in deformation of the stamp that would allow the polymer in micro-wells 206 to be transferred to the slide);

(iii) place the weight, stamp, and slide on a hot plate at about 110° C. for about ten seconds, and then remove the polymer-coated stamp from the glass slide (see FIG. 2c) leaving the excess polymer 211 that coated micro-pillars 204 on the surface of the slide (note: the polymer deposited on the slide in this step is no longer needed and this slide may be discarded or recycled after this step);

(iv) using a cotton swab or other suitable applicator, brush a 1.5 wt % aqueous solution of polyvinyl alcohol (PVA) onto the surface of a second glass slide (new substrate 212) to form a thin film which will serve as sacrificial layer 210;

(v) place stamp 202 on substrate 212 with the polymer-coated face touching the surface of the slide and sacrificial layer 210, and for about five seconds place a solid weight or other suitable compression means on top of the stamp (creating a pressure of greater than about 2.5 kPa) to push the polymer in micro-wells 206 onto substrate 212 (see FIG. 2d);

(vi) place the weight, stamp, and slide on a hot plate at about 110° C. for about ten seconds, and then remove the polymer-coated stamp from the glass slide (see FIG. 2c) leaving the polymer that coated micro-wells 206 on the surface of the slide attached to sacrificial layer 210 (see FIG. 2e);

(vii) place substrate 212 into water-filled reservoir 216 to dissolve sacrificial layer 210 and release microparticles 214 into solution (see FIG. 2f); and (optionally)

(viii) retrieving microparticles from solution by means of desiccation, filtration, or any other suitable method.

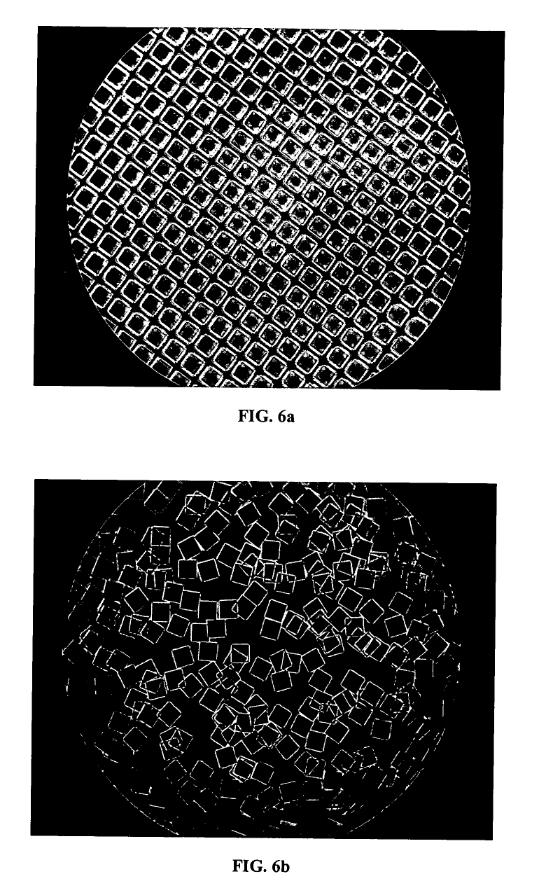

With reference to FIGS. 6a-b, the stamp used in this embodiment includes 40 um-wide square micro-wells separated by 10 um-wide ridges which are about 1.4 μm height. The microparticles created by this exemplary method have an average thickness of about 130 nm; however, the rims or outer edges of these microparticles may be as thick as about 300 nm to 600 nm. FIG. 6a is an optical micrograph of microparticles 214 as they appear on the surface of substrate 212. The square-like shape of the microparticles is clearly evident in FIG. 6a. FIG. 6b is an optical micrograph of microparticles 214 released into a solution of water after sacrificial layer 212 has been dissolved.

III. “Discontinuous Wetting” Method

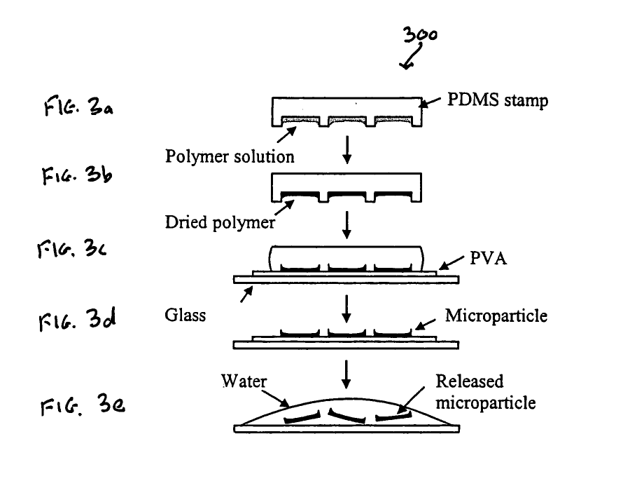

With reference to FIGS. 3a-e, a third embodiment of this invention, microfabrication system 300, uses a stamp 302, a substrate 312, and a water-soluble sacrificial layer 310 to create polymer microparticles 314. Utilizing system 300, microparticles 314 are fabricated according to the following exemplary method:

(i) apply 10 wt % poly(lactic-glycolic)acid (PLGA)/dimethyl sulfoxide (DMSO) solution to stamp 302 to fill only the micro-well features (see FIG. 3a);

(ii) evaporate the solvent (DMSO) under vacuum overnight, leaving PLGA solid polymer 308 in the micro-well features on the face of the stamp (see FIG. 3b);

(iii) using a cotton swab or other suitable applicator, brush a 1.5 wt % aqueous solution of polyvinyl alcohol (PVA) onto the surface of a glass slide (new substrate 312) to form a thin film which will serve as sacrificial layer 310;

(iv) place stamp 302 on substrate 312 with the polymer-coated face touching the surface of the slide and sacrificial layer 310, and for about five seconds place a solid weight or other suitable compression means on top of the stamp (creating a pressure of greater than about 2.5 kPa) to push the polymer in micro-wells 306 onto substrate 312 (see FIG. 3c);

(v) place the weight, stamp, and slide on a hot plate at about 110° C. for about ten seconds, and then remove the polymer-coated stamp from the glass slide leaving the polymer that coated the micro-wells 306 on the surface of the slide attached to sacrificial layer 310 (see FIG. 3d);

(vi) place substrate 312 into water-filled reservoir 316 to dissolve sacrificial layer 310 and release microparticles 314 into solution (see FIG. 3e); and (optionally)

(vii) desiccate, filter, or use other conventionally accepted methods to retrieve microparticles from solution.

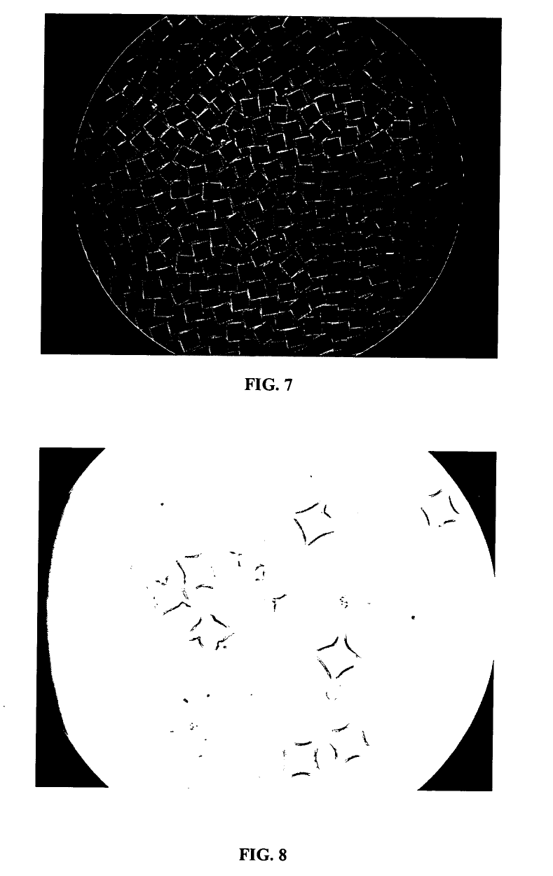

With reference to FIG. 7, the stamp used in this embodiment includes 40 um-wide square micro-wells separated by 10 um-wide ridges which are about 1.4 μm height. FIG. 7 is an optical micrograph of microparticles 314 released into a solution of water immediately after sacrificial layer 312 has been dissolved, still floating loosely above their original positions on the substrate. This technique can be used for solution casting as described above with the appropriate solvent/stamp combination, or also for casting and curing a pre-polymer solution such as methacrylic acid (MAA) for the formation of cross-linked microparticles (PMAA, a hydrogel).

IV. “Multi-Layer” Method

With reference to FIGS. 4a-h, a fourth embodiment of this invention, microfabrication system 400, uses a stamp 402, a substrate 412, and a water-soluble sacrificial layer 410 to create polymer microparticles 414. Utilizing system 400, microparticles 414 are fabricated according to the following exemplary method:

(i) stamp 402 is dipped into a 2.5 wt % PPMA/acetone solution to form a thin, continuous layer 408 of PPMA on the face of the stamp (see FIG. 4a) and covering its contours, i.e., micro-pillars 404 and micro-wells 406;

(ii) place the polymer-coated stamp on a glass slide 412 (see FIG. 4b) and apply pressure of about 550 Pa to the stamp using a solid weight or other suitable compression means to induce a full conformal contact between the polymer on the raised regions of the stamp and the glass slide across the entire face of the stamp (note: care should be taken to not apply excessive pressure resulting in deformation of the stamp that would allow the polymer in micro-wells 406 to be transferred to the slide);

(iii) place the weight, stamp, and slide on a hot plate at about 110° C. for about ten seconds, and then remove the polymer-coated stamp from the glass slide leaving the excess polymer 411 that coated micro-pillars 404 on the surface of the slide (note: the polymer deposited on the slide in this step is no longer needed and this slide may be discarded or recycled after this step);

(iv) brush fully sulfonated polyaniline (FSPAN)/DMSO solution onto stamp 402 to form spots of solution within the microwell features 406 on top of the previously deposited PPMA;

(v) evaporate DMSO solvent in vacuum overnight, leaving solid polymer FSPAN on top of PPMA within the microwells 406 (see FIG. 4c);

(vi) dip stamp 402 into a 2.5 wt % PPMA/acetone solution to form a thin, continuous layer 408 of PPMA on the face of the stamp (see FIG. 4d) that is bonded to the first layer of PPMA (see FIG. 4d);

(vii) repeat steps (ii) and (iii) to remove excess polymer 411 that coats the micro-pillars 404 onto the surface of the slide (see FIG. 4e);

(viii) using a cotton swab, brush a 1.5 wt % aqueous solution of polyvinyl alcohol (PVA) onto the surface of a new glass slide (new substrate 412) to form a thin film which will serve as sacrificial layer 410;

(ix) place stamp 402 on substrate 412 with the polymer-coated face touching the surface of the slide and sacrificial layer 410, and for about five seconds place a solid weight or other suitable compression means on top of the stamp (creating a pressure of greater than about 2.5 kPa) to push the polymer in micro-wells 406 onto substrate 412 (see FIG. 4f);

(x) place the weight, stamp, and slide on a hot plate at about 110° C. for about ten seconds, and then remove the polymer-coated stamp from the glass slide leaving the p olymer that coated micro-wells 406 on the surface of the slide attached to sacrificial layer 410 (see FIG. 4g);

(xi) place substrate 412 into water-filled reservoir 416 to dissolve sacrificial layer 410 and release microparticles 414 into solution (see FIG. 4h); and (optionally)

(xii) desiccate, filter, or use other conventionally accepted methods to retrieve microparticles from solution.

With reference to FIG. 8, the stamp used in this embodiment includes 40 um-wide square micro-wells separated by 10 um-wide ridges which are about 1.4 μm height. The microparticles created by this exemplary method demonstrate the multi-layer properties through the swelling of the confined FSPAN layer which is completely encapsulated between the two PPMA layers. FIG. 8 is an optical micrograph of microparticles 414 released into a solution of water after sacrificial layer 412 has been dissolved and the interior FSPAN layer has swollen. This technique can be used to produce microparticles of any multitude of layers for added functionality, so long as the cumulative thickness of the microparticles is less than the micro-well depth on the PDMS stamp.

All embodiments of the system and method of the present invention enable microfabrication of geometrically uniform microparticles over relatively large surface areas on the substrate. Optical profilometry can be employed to confirm that these microparticles have the same lateral sizes as the stamp structures for both the micro-pillar method and micro-well methods. Optical profilometry can also be used to confirm that microparticles made with the micro-pillar method are typically thicker in the center portion of the particle, while the microparticles made with micro-well method typically include a thin central portion but have a thicker rim portion.

While the exemplary methods disclosed herein include the use of stamps with square-like structures, stamps or other templates having any number of different geometries can be used to create polymer microparticles. Thus, polymer microparticles having any variety of lateral shapes can be produced with these methods provided that a continuous film of polymer is formed on the face of the stamp such that it covers the micro-structures or micro-contours of the stamp. In some embodiments that utilize different stamps or templates, the concentration of the polymer solution for dip coating may have to be adjusted to achieve optimal film formation. In general, the thickness of the film and of the resultant microparticles, is proportional to the concentration of the solution. Thus, for different polymers or combinations of polymers, optimal concentrations should be determined empirically. Likewise, polymers other than those described in the exemplary methods will have different thermal and cross-linking properties; therefore, system parameters such as temperatures and exposure times may need to be adjusted accordingly.

The systems and methods disclosed basically fall into to broad categories, namely the “micro-pillar” technique and the “micro-well” technique. Although closely related, each technique has its own particular applications and advantages. For example, the micro-pillar printing technique, is essentially a one-step process which is simplistic and relative easy to perform. This one-step process may be repeated using the same stamp and the same substrate to create polymer structures having multiple layers. Each new layer added to the first layer of polymer may include the same or different polymer(s) and the same or different shapes, patterns, geometries, or other desired characteristics. The micro-well printing technique is essentially a two-step process that includes an additional printing step to remove unneeded polymer film on the ridges of the stamp before printing out the microparticles on the substrate.

The micro-well printing technique can also be used to fabricate multi-layered microparticles by filling the micro-wells multiple times and transferring the polymer to the substrate to create composite microparticles. The discontinuous wetting and the multi-layered method described above are embodiments of the present invention that incorporate the micro-well technique. Advantageously, the micro-well method may also be performed partially in the absence of elevated temperature, which is only needed to remove polymer between the micro-wells in the first printing. The second printing, which transfers polymer in the micro-wells onto the sacrificial layer, can be carried out at room temperature simply by making the sacrificial layer tacky, which is easily achieved through a brief exposure of a dry PVA layer to hot water vapor.

It should also be noted, that while the sacrificial layer of material is included as a component in the described system and methods, all of the methods described herein can be performed without this sacrificial layer to create microparticles that remain attached to the substrate material following the various printings.

While the above description contains many specificities, these should not be construed as limitations on the scope of the invention, but rather as exemplification of certain preferred embodiments. Numerous other variations of the present invention are possible, and is not intended herein to mention all of the possible equivalent forms or ramifications of this invention. Various changes may be made to the present invention without departing from the scope or spirit of the invention.

What is claimed:

1. A system for fabricating polymer microparticles, comprising:
(a) a stamp, wherein said stamp further comprises micro-structures on at least one side of said stamp for receiving a layer of said polymer;
(b) a substrate; and
(c) a layer of dissolvable material covering said substrate.

2. The system of claim 1, further comprising a compression means for compressing said stamp against said substrate.

3. The system of claim 1, further comprising a solvent for dissolving said layer of dissolvable material.

4. The system of claim 3, further comprising a reservoir for said solvent.

5. The system of claim 1, wherein said polymer is polypropyl methacrylate, polylactic-co-glycolic acid, polycaprolactone, polymethyl methacrylate, or polystyrene.

6. The system of claim 1, wherein said stamp is a polydimethyl siloxane stamp.

7. The system of claim 1, wherein said micro-structures further comprise a plurality of micro-pillars.

8. The system of claim 1, wherein said micro-structures further comprise a plurality of micro-wells.

9. The system of claim 1, wherein said substrate is a glass slide.

10. The system of claim 1, wherein said layer of dissolvable material further comprises polyvinyl alcohol.

11. The system of claim 1, wherein said layer of dissolvable material further comprises a water soluble ink, glucose, chitosan, or polyethylene glycol.

12. A method for creating polymer microparticles, comprising the steps of:
(a) applying a thin, continuous layer of polymer to the contoured side of a stamp, wherein said contours include individual protruding microstructures;
(b) covering the surface area of a substrate with a solution of dissolvable material and allowing said solution to dry on the surface of said substrate;
(c) placing said stamp, polymer coated side down, on said substrate such that said protruding microstructures make contact with said substrate;
(d) applying compression means to the side of said stamp opposite the side that is in contact with said substrate;
(e) placing said substrate, stamp and said compression means on a heat source for a predetermined period of time;
(f) removing said stamp from said substrate; and
(g) placing said substrate in a solvent that will dissolve said dissolvable material and release said polymer microparticles from said substrate.

13. The method of claim 12, further comprising the step of desiccating or filtering said solvent to recover said microparticles from solution.

14. The method of claim 12, wherein said polymer is polypropyl methacrylate, polylactic-co-glycolic acid, polycaprolactone, polymethyl methacrylate, or polystyrene.

15. The method of claim 12, wherein said stamp is a polydimethyl siloxane stamp.

16. The method of claim 12, wherein said substrate is a glass slide.

17. The method of claim 12, wherein said dissolvable material further comprises polyvinyl alcohol.

18. The method of claim 12, wherein said layer of dissolvable material further comprises a water soluble ink, glucose, chitosan, or polyethylene glycol.

19. The method of claim 12, wherein said solvent is water.

20. A method for creating polymer microparticles, comprising the steps of:
(a) applying a thin, continuous layer of polymer to the contoured side of a stamp, wherein said contours include individual recesses;
(b) placing said stamp, polymer-coated side down, on a first substrate;
(c) applying compression means to said stamp sufficient to transfer polymer on the regions between said individual recesses to said first substrate but insufficient to transfer polymer in said individual recesses to said first substrate;
(d) placing said first substrate, stamp and said compression means on a heat source for a predetermined period of time;
(e) removing said stamp from said first substrate and discarding said first substrate;
(f) covering the surface area of a second substrate with a solution of dissolvable material and allowing said solution to dry on the surface of said substrate to form a layer;
(g) placing said stamp, polymer coated side down, on said second substrate;
(h) applying compression means to said stamp sufficient to transfer polymer in said individual recesses to said second substrate;
(i) placing said second substrate, stamp and said compression means on a heat source for a predetermined period of time;
(j) removing said stamp from said second substrate; and
(k) placing said second substrate in a solvent that will dissolve said dissolvable material and release said polymer microparticles from said second substrate.

21. The method of claim 20, further comprising the step of desiccating or filtering said solvent to recover said microparticles from solution.

22. The method of claim 20, wherein said polymer is polypropyl methacrylate, polylactic-co-glycolic acid, polycaprolactone, polymethyl methacrylate, or polystyrene.

23. The method of claim 20, wherein said stamp is a polydimethyl siloxane stamp.

24. The method of claim 20, wherein said substrates are glass slides.

25. The method of claim 20, wherein said dissolvable material further comprises polyvinyl alcohol.

26. The method of claim 20, wherein said dissolvable material further comprises a water soluble ink, glucose, chitosan, or polyethylene glycol.

27. The method of claim 20, wherein said solvent is water.

28. A method for creating polymer microparticles, comprising the steps of:
(a) applying a thin layer of polymer and a first solvent only to the individual recesses on the contoured side of a stamp;
(b) allowing said first solvent to evaporate;
(c) covering the surface area of a substrate with a layer of dissolvable material and allowing said solution to dry on the surface of said substrate;
(d) placing said stamp, polymer coated side down, on said substrate;
(e) applying compression means to said stamp sufficient to transfer polymer in said individual recesses to said substrate leaving said polymer attached to said layer of dissolvable material;
(f) removing said stamp from said substrate; and
(g) placing said substrate in a second solvent that will dissolve said dissolvable material and release said polymer microparticles from said second substrate.

29. The method of claim 28, further comprising the step of desiccating or filtering said second solvent to recover said microparticles from solution.

30. The method of claim 28, wherein said polymer is polypropyl methacrylate, polylactic-co-glycolic acid, polycaprolactone, polymethyl methacrylate, or polystyrene.

31. The method of claim 28, wherein said stamp is a polydimethyl siloxane stamp.

32. The method of claim 28, wherein said dissolvable material further comprises polyvinyl alcohol, a water soluble ink, glucose, chitosan, or polyethylene glycol.

33. The method of claim 20, wherein said second solvent is water.

34. A method for creating polymer microparticles, comprising the steps of:
(a) applying a thin, continuous layer of a first polymer to the contoured side of a stamp, wherein said contours include individual recesses;
(b) removing said polymer on the face of said stamp between said individual recessed areas;
(c) applying a solution of a material and a first solvent to said individual recesses on top of said first polymer;
(d) allowing said first solvent to evaporate leaving said material in said individual recesses;
(e) applying a thin, continuous layer of a second polymer to the contoured side of a stamp, wherein said contours include individual recesses;
(f) removing said polymer on the face of said stamp between said individual recessed areas;
(g) covering the surface area of a second substrate with a solution of dissolvable material and allowing said solution to dry on the surface of said substrate;
(h) placing said stamp, polymer coated side down, on said second substrate;
(i) applying compression means to said stamp sufficient to transfer polymer in said individual recesses to said second substrate;
(j) placing said second substrate, stamp and said compression means on a heat source for a predetermined period of time;
(k) removing said stamp from said second substrate; and
(l) placing said second substrate in a second solvent that will dissolve said dissolvable material and release said polymer microparticles from said second substrate.

35. The method of claim 34, further comprising the step of desiccating or filtering said second solvent to recover said microparticles from solution.
